(19) United States
(12) Patent Application Publication
(10) Pub. No.: US 2021/0180080 A1
(43) Pub. Date: Jun. 17, 2021

(54) USE OF ALS MUTANT PROTEIN AND THE GENE THEREOF IN PLANT BREEDING BASED ON GENE EDITING TECHNOLOGY

(71) Applicant: JIANGSU ACADEMY OF AGRICULTURAL SCIENCES, Nanjing (CN)

(72) Inventors: Jie YANG, Nanjing (CN); Fangquan WANG, Nanjing (CN)

(21) Appl. No.: 16/981,549

(22) PCT Filed: Apr. 17, 2019
(86) PCT No.: PCT/CN2019/082973

§ 371 Date: Sep. 16, 2020

(30) Foreign Application Priority Data
Sep. 6, 2018 (CN) 201811038025.5

(51) Int. Cl. C12N 15/82; C12Q 1/6895; C12N 9/10

(52) U.S. Cl. CPC C12N 15/8274; C12Q 1/6895; C12Y 202/01006; C12N 2310/20; C12N 15/8213; C12Q 2600/13; C12N 2800/80; C12N 9/1022

(57) ABSTRACT

It discloses a rice ALS mutant protein, a mutant gene, and uses thereof, wherein a mutation corresponding to an amino acid at position 628 of the amino acid sequence of the rice ALS is present in the amino acid sequence of the rice ALS protein. The present invention further discloses a breeding method for creating herbicide resistant rice using gene editing. The present invention uses CRISPR/Cas9 gene editing technology for the first time to edit ALS genes. Through the screening of offspring, a new T-DNA free variety having herbicide resistance stably inherited can be obtained in the T₂ generation, and the basic agronomic characteristics of the new variety have no obvious change.

### FIELD OF TECHNOLOGY

The invention belongs to a field of crop genetic breeding and new resource innovation for crop herbicide resistance, and specifically relates to the use of ALS mutant protein and the gene thereof in plant breeding based on gene editing technology.

### BACKGROUND

With the development of new urbanization and modern agriculture in China, the simple cultivation of rice production has become more and more popular, and methods such as machine transplanting and direct seeding have become a development trend. However, it is easy to breed weeds and weedy rice in direct seeding rice fields, which seriously affects the growth, yield and quality of rice. The high cost of manual and mechanical weeding restricts the development of rice production in the direction of high yield, high efficiency and low cost, and is not conducive to the development of modern agriculture. Spraying herbicides is an effective way to control weeds and weedy rice harm.

The biosynthesis of branched-chain amino acids (valine, leucine and isoleucine) in plants and microorganisms requires the co-catalysis of four enzymes, acetolactate synthase (ALS), ketol-acid reductoisomerase, dihydroxyavid dehydratase and branched-chain amino acidtransaminase. Acetolactate synthase is the key enzyme in the first stage of the biosynthesis process, which catalyzes 2 molecules of pyruvate to produce acetolactate and carbon dioxide in the synthesis of valine and leucine, and catalyzes 1 molecule of pyruvate and 1 molecule of α-butyric acid to produce 2-glyoxy-2-hydroxybutyric acid and carbon dioxide in the synthesis of isoleucine. ALS inhibitor herbicides inhibit the ALS enzyme activity in plants, thereby preventing the synthesis of branched-chain amino acids, leading to the destruction of protein synthesis, hindering DNA synthesis during cell division, thereby stopping the mitosis of the plant cell at the S phase (DNA synthesis phase) of the G1 stage and the M phase of the G2 stage, which interferes with the synthesis of DNA, therefore the cell cannot complete the mitosis, thereby stopping the growth of the plant, and ultimately leading to the death of the individual plant.

Acetolactate synthase (ALS) (also known as acetohydroxy acid synthase, AHAS; EC 4.1.3.18) inhibitor herbicides target ALS to cause weed death, mainly including 13 classes of compounds such as Sulfonylureas (SU), imidazolinones (IMO, Triazolopyrimidines (TP), Pyrimidinylthio(or oxy)-benzoates (PTB; pyrimidinyl-carboxyherbicides; PCs) and Sulfonylamino-carbonyltriazolinones (SCT). Acetolactate synthase present in the process of plant growth, can catalyze pyruvate to acetolactate with high specificity and high catalytic efficiency, leading to the biosynthesis of branched chain amino acids.

imidazolinone herbicides are a kind of highly effective, broad-spectrum and low toxicity herbicides developed by the American Cyanamid Company. There are currently 6 commercial products, including: imazapyr, imidazolium nicotinic acid, imazamethabenz, imazaquin, imazamox and imazameth. imidazolium nicotinic acid, also known as imazethapyr, is an efficient herbicide commonly used in soybean fields, which can effectively control annual gramineous weeds and broad-leaved weeds. However, these herbicides also cause phytotoxicity to crops that generally do not have the characteristics of herbicide resistance (tolerance), which greatly limits their use time and space, for example, it is necessary to use herbicides a period of time before planting to prevent crops from suffering from phytotoxicity. Cultivating herbicide resistant (tolerant) crop strains can reduce crop phytotoxicity and broaden the range of herbicide use.

Currently, the known herbicide resistance mutation sites of ALS genes in rice include Gln 25, Gly 95, Ala 96, Gln 113, Ala 122, Ser 160, Pro 171, Ala 179, Ala 237, Asn 350, His 367, Lys 390, Trp 548, Ser 627 and Leu 636. The herbicide resistance level of ALS mutants is related to the position of ALS amino acid mutation, and also related to the type of amino acid after mutation and the number of amino acid mutation. Therefore, creating and screening new types of herbicide resistant mutations is conducive to enriching the genetic diversity of herbicide resistant genes and providing genetic germplasm for the cultivation of new rice strains.

Currently, screening of new genetic germplasm for herbicide resistance is mainly through chemical mutagenesis. It is well known that the frequency of chemical mutations is low and the initial investment is large. It takes 2-3 years to screen and obtain a resistant and stable variety. At the same time, chemical mutagenesis may cause multiple gene mutations in wild-type varieties. It needs to be improved through hybridization to delete undesirable genes in production. The target genes are introduced into the background of excellent varieties using conventional breeding methods, mainly through cross, backcross, multiple cross, step cross, etc. to introduce the target gene into the background parent. For quality traits, backcrossing is a commonly used method. Generally, 4-6 generations of backcrossing are required, with additional at least 3-5 generations for such as self-crossing homozygous and the like. Therefore, it takes at least 3-5 years to improve one trait and be able to play a role in production.

Compared with traditional breeding, gene editing breeding is more efficient. Because of the high efficiency of genetic transformation mediated by Agrobacterium in japonica rice varieties, we found that 10 transformants of T₀ generation can generally screen 3-5 homozygous target alleles that are edited at the same time, that is, the phenotype can be observed in the T₀ generation, such as fragrance, dark endosperm caused by low amylose content, early heading date, etc. Utilizing the seeds produced by an individual plant of the T₀ generation in the T₁ generation, generally 5 plants with the hygromycin and Cas9 genes deleted can be selected out of 100 plants, and 5 seedlings can be propagated. Generally, each plant can harvest 500 seeds. One plant can propagate 400 plants, which can propagate about 20 kilograms of conventional japonica seeds, which are enough for quality analysis and even various experiments such as trials, demonstrations and the like. Even if clonal mutations occur during tissue culture in the T₀ generation, the use of genome-edited variety and wild-type hybridization can delete undesirable mutations.

The use of genome editing technology can accurately create the desired target gene, which cannot be achieved by conventional breeding methods. Conventional breeding can only select the germplasm with target genes from variety germplasms, and then improve existing varieties through cross-backcrossing and other means. Generally, the agronomic characteristics of germplasm variety are poor, so the cycle of genetic improvement is long. But the genome editing technology can directly use rice varieties with excellent agronomic traits and widely promoted in production as background variety to edit target genes. At present, the Jiangsu Academy of Agricultural Sciences has constructed a multi-gene editing vector for heading date-related genes, using Wuyunjing 24 and Nanjing 9108 widely promoted in Jiangsu as the genetic transformation background variety, from which new variety with heading dates of more than 60 days, more than 70 days, and more than 80 days are obtained with exogenous marker genes deleted. Compared with the wild type, the heading period is earlier and exhibits a gradient distribution, which can expand the planting range of high-quality varieties, accelerate the improvement process of high-quality varieties, greatly save breeding costs, and highlight the vitality and creativity of gene editing molecular breeding.

Genotype selection can be performed with the help of molecular marker assisted selection breeding, and the heterozygous and homozygous genotypes of the target trait gene can be screened, and the homozygous process of the target gene can be accelerated. Therefore, the development of gene functional markers is beneficial to speed up the breeding process. Most of the functional mutations of ALS genes are single base mutations, which can be targeted to design restriction target markers, but the process is relatively cumbersome. The development of allele-specific PCR can distinguish susceptibleness resistant genotypes through two cycles of PCRs.

It is reported that mutations in Trp 548 and Ser 627 can make proteins more resistant to ALS inhibitor herbicides, but different types of mutations at these sites still have different effects. Researchers have successfully achieved the amino acid variations at position 548 from tryptophan to leucine and at position 627 from serine to isoleucine by using gene substitution technology. However, this method is based on the reported base variation and cannot create new types of variation

At present, scientists and technicians who want to obtain an herbicide resistant rice variety or new genes need to use chemical or radiation mutagenesis. The workload is very large and the effect is very unsatisfactory. Moreover, most of the herbicide resistant ALS proteins obtained are the variants that have been reported.

However, with conventional chemical mutation and conventional trans-breeding breeding, the breeding period must be at least 4-6 years. And at present, there is no relevant report on the use of gene editing technology to site-directed mutation of ALS gene in rice varieties to create new herbicide resistant alleles.

### SUMMARY

Objective of the invention: The technical problem to be solved by the present invention is to provide a rice ALS mutant protein and a nucleic acid or gene thereof.

The technical problem to be solved by the present invention is to provide an expression cassette, recombinant vector or cell.

The technical problem to be solved by the present invention is to provide a rice ALS mutant protein, a nucleic acid or gene, and use of the expression cassette, recombinant vector or cell in herbicide resistance of plants.

The technical problem to be solved by the present invention is to provide a breeding method for creating herbicide resistant rice using gene editing. The present invention performs site-directed mutations on ALS genes of rice varieties by the gene editing technology for the first time to create new herbicide resistant alleles, delete T-DNA sequences, and obtain stable genetic resistance lines in only about 2 years, the breeding period of which is at least 2-4 years earlier than by chemical mutation and conventional transfer breeding. Therefore, gene editing molecular breeding has the advantages of precision and high efficiency that conventional breeding does not possess, and has broad application prospects.

The technical problem to be solved by the present invention is to provide a primer set for identifying the gene or nucleic acid.

The technical problem to be solved by the present invention is to provide use of the gene or nucleic acid, the primer set in the identification and breeding of an herbicide resistant variety.

Technical solution: In order to solve the above technical problems, the technical solution adopted by the present invention is as follows: A rice ALS mutant protein, wherein a mutation corresponding to an amino acid at position 628 of the amino acid sequence of the rice ALS is present in the amino acid sequence of the rice ALS protein.

In particular, the present invention reports for the first time that the amino acid at position 628 is mutated from glycine to tryptophan for herbicide resistance. The mutation of the amino acid at position 628 of the present invention may also include 21 variant types such as glutamic acid, aspartic acid, tryptophan, alanine, valine, leucine, isoleucine, proline, phenylalanine, tyrosine, serine, threonine, cysteine, methionine, asparagine, glutamine, lysine, arginine, histidine and stop codons and the like. However, further studies are needed to confirm whether other variations or early termination of the above amino acids affect the activity of acetolactate synthase, physiological functions and herbicide resistance.

The rice ALS mutant protein of the present invention comprises:

(a) an amino acid sequence thereof as shown in SEQ ID NO: 2; or

(b) a protein derived from (a) in which the amino acid sequence in (a) has been substituted and/or deleted and/or added one or several amino acids and has acetolactate synthase activity.

The summary of the invention further comprises a nucleic acid or a gene, which encodes the mutant protein.

Wherein, the nucleic acid or gene comprises:

(a) encoding the mutant protein; or

(b) a nucleotide sequence that hybridizes with the defined nucleotide sequence of (a) under stringent conditions and encodes a protein with acetolactate synthase activity; or

(c) a nucleotide sequence thereof as shown in SEQ ID NO:1.

The summary of the invention further comprises an expression cassette, recombinant vector or cell, which contains the nucleic acid or gene.

The summary of the invention further comprises use of the rice ALS mutant protein, the nucleic acid or gene, the expression cassette, recombinant vector or cell in herbicide resistance of plants.

The summary of the present invention further comprises a method for obtaining an herbicide resistant plant, which comprises the following steps:

1) making the plant contain the nucleic acid or gene; or

2) making the plant express the rice ALS mutant protein.

The summary of the present invention further comprise a breeding method for creating herbicide resistant rice using gene editing, which comprises the following steps:

1) cloning ALS gene and designing a target site for gene editing;

2) construction of CRISPR/Cas9 gene editing vector containing a target fragment;

3) obtaining herbicide resistant rice with the ALS mutant protein, or with the nucleic acid or the gene.

Wherein, the method for constructing the CRISPR/Cas9 gene editing vector containing the target fragment in the step 2) is as follows:

A) preparation of a target adaptor: a adaptor primer is dissolved with TE to obtain a stock solution, which is placed at 90° C. for 30 s after dilution and then cooled at room temperature to complete annealing, thereby obtaining the target adaptor;

B) preparation of an sgRNA ligation product: the sgRNA ligation product is obtained by PCR amplification using a pYLsgRNA-OsU3 intermediate vector, the target adaptor, DNA ligase, and BsaI;

C) sgRNA expression cassette amplification: the sgRNA ligation product is subjected to the first cycle of PCR amplification by a primer combination of forward primer U-F and reverse primer sgRNA-R to obtain the first cycle of PCR product, which is then subjected to the second cycle of PCR after dilution by Uctcg-B1 and gRcggt-BL as amplification primers to obtain a PCR product which is the sgRNA expression cassette;

D) the sgRNA expression cassette is ligated to a CRISPR/Cas9 expression vector to obtain a ligation product;

E) the ligation product from step D) is transformed into E. coli by heating shock to obtain recombinant bacteria, and positive plasmids are extracted from the verified bacterial solution containing a target brand.

Wherein, the obtaining method of the herbicide resistant rice in step 3) is as follows: the CRISPR/Cas9 gene editing vector containing the target fragment from step 2) is transformed into Agrobacterium EHA105 to obtain a T₀ generation transgenic plant with herbicide resistance, for which the sequence is amplified and identified by primers ALST-F and ALST-R to obtain a plant with the mutant protein or the nucleic acid or gene.

Wherein, the breeding method further comprises that a T-DNA vector including the hygromycin phosphotransferase gene HPT and the nuclease gene Cas9 of a T₁ generation plant containing the target allele double mutation from the T₀ generation transgenic plant with herbicide resistance is deleted.

Wherein, the deletion of the T-DNA vector involves simultaneous detection for HPT gene and Cas9 gene in the T₁ generation plant containing the target allele double mutation, which is repeated for multiple times to obtain a T₁ generation individual plant without the two genes after screening, referred as a target plant.

Wherein, the detection for HPT gene involves PCR amplification using the genomic DNA of the T₁ generation plant with the target allele double mutation as a template, and hyg283-F and hyg283-R as primers, and meanwhile, the detection for Cas9 gene involves PCR amplification using the genomic DNA of the T₁ generation plant with the target allele double mutation as a template, and Cas9T-F and Cas9T-R as primers, and absence of both the HPT gene and the Cas9 gene, indicates that the T-DNA is successfully deleted.

The summary of the invention further comprises a primer set for identifying the gene or nucleic acid, the primer set is ALS4 with sequences as shown in SEQ ID NO: 6 and SEQ ID NO: 7, and/or ALS6 with sequences as shown in SEQ ID NO: 8 and SEQ ID NO: 9.

The summary of the invention further comprises use of the ALS mutant gene or nucleic acid and the primer set in the identification and breeding of an herbicide resistant variety.

Beneficial effects: Compared with the prior art, the present invention has the following advantages:

1) The present invention edits the ALS gene by CRISPR/Cas9 gene editing technology for the first time. Through the screening of offspring, a new T-DNA free variety having herbicide resistance stablely inherited can be obtained in the T₂ generation. There is no significant change in agronomic traits. Compared with chemical mutagenesis, cross-breeding and other breeding methods, gene editing directed improvement molecular breeding technology is fast, accurate, and efficient. And the use of gene function markers for genotype selection will greatly improve breeding efficiency and greatly accelerate the breeding process.

2) Based on the base variation between wild-type and mutant at position 1882 of the ALS gene, the present invention has developed a specific distinction between wild-type (base 1882 of ALS gene is G) and mutant (base 1882 of ALS gene is T) molecular markers ALS4 and ALS6 can be used in molecular marker assisted selection breeding.

3) After applying 210 g(a.i.)hm⁻² “Imazethapyr” (equivalent to 3 times the recommended concentration) to seedlings with 1-2 leaves of rice varieties bred by the gene editing technology of the invention, the plants still grow and develop normally, but the seedlings with 1-2 leaves of wild-type rice appear to die in whole 14 days after applying 210 g(a.i.)hm⁻² “Imazethapyr” (equivalent to 3 times the recommended concentration, 70 g(a.i.)hm⁻²).

4) After applying 240 g(a.i.)hm⁻² “Bailongtong” (equivalent to 1 time the recommended concentration, 240 g(a.i.)hm⁻²) to seedlings with 1-2 leaves of rice varieties bred by the gene editing technology of the invention, the plants still grow and develop normally, but the seedlings with 1-2 leaves of wild-type rice appear to die in whole 14 days after applying 240 g(a.i.)hm⁻² “Bailongtong” (equivalent to 1 time the recommended concentration, 240 g(a.i.)hm⁻²).

### BRIEF DESCRIPTION OF THE DRAWINGS

FIG. 1 Base variation of transgenic plants;

FIG. 2 Resistant rice mutants obtained by herbicide screening; WT is Nanjing 9108, and A3, A5, A9, A24 and A51 are T₁ generation transgenic lines;

FIG. 3A HPT gene detection results of T₁ generation plants; M is DL2000 molecular marker, 1-18 are T₁ generation transgenic plants, 19 is a positive control with plasmid as template, 20 is a negative control of Nanjing 9108 template;

FIG. 3B Cas9 gene detection results of T₁ generation plants; M is DL2000 molecular marker, 1-18 are T₁ generation transgenic plants, 19 is a positive control with plasmid as template, 20 is a negative control of Nanjing 9108 template;

FIG. 4A The treatment results of the mutant variety imazethapyr (wild-type): 1-4 are sprayed with imazethapyr at the concentration of 0, 210, 700 or 1400 g(a.i.) hm⁻², respectively;

FIG. 4B The treatment results of the mutant variety imazethapyr (the mutant): 1-4 are sprayed with imazethapyr at the concentration of 0, 210, 700 or 1400 g(a.i.) hm⁻², respectively;

FIG. 5A The treatment results of the mutant variety Bailongtong (wild-type): 1-4 are sprayed with Bailongtong at the concentration of 0, 240, 2400 or 4800 g(a.i.) hm⁻², respectively;

FIG. 5B The treatment results of the mutant variety Bailongtong (the mutant): 1-4 are sprayed with Bailongtong at the concentration of 0, 240, 2400 or 4800 g(a.i.) hm⁻², respectively;

FIG. 6A Comparison of agronomic traits between the mutant and the wild-type; represent plant height;

FIG. 6B Comparison of agronomic traits between the mutant and the wild-type; represent effective panicle;

FIG. 6C Comparison of agronomic traits between the mutant and the wild-type; represent panicle length;

FIG. 6D Comparison of agronomic traits between the mutant and the wild-type; represent number of grains per panicle;

FIG. 6E Comparison of agronomic traits between the mutant and the wild-type; represent seed setting rate;

FIG. 6F Comparison of agronomic traits between the mutant and the wild-type; represent thousand-grain weight;

FIG. 7A Development of ALS628W functional marker (wild type): M is DL2000 molecular marker, 1-13 are molecular markers ALS1˜ALS13, respectively;

FIG. 7B Development of ALS628W functional marker (mutant): M is DL2000 molecular marker, 1-13 are molecular markers ALS1˜ALS13, respectively;

FIG. 8A ALS628W functional marker detection varieties (ALS4): M is a DL2000 molecular marker, and 1-27 are Nanjing 9108 mutant, Nanjing 9108 wild type, Nipponbare, Huang Huazhan, 9311, Lianjing 7, Suxiu 867, Zhendao 88, Zhendao 99, Huaidao 5, Changnongjing 8, Nanjing 44, Nanjing 45, Nanjing 46, Nanjing 49, Nanjing 51, Nanjing 47, Nanjing 5055, Suken 118, Wuyunjing 24, Wuyunjing 27, Wuyunjing 29, Xudao 8, Xudao 9, Yangyujing 2, Huajing 5, Yandao 16, respectively;

FIG. 8B ALS628W functional marker detection varieties (ALS6): M is a DL2000 molecular marker, and 1-27 are Nanjing 9108 mutant, Nanjing 9108 wild type, Nipponbare, Huang Huazhan, 9311, Lianjing 7, Suxiu 867, Zhendao 88, Zhendao 99, Huaidao 5, Changnongjing 8, Nanjing 44, Nanjing 45, Nanjing 46, Nanjing 49, Nanjing 51, Nanjing 47, Nanjing 5055, Suken 118, Wuyunjing 24, Wuyunjing 27, Wuyunjing 29, Xudao 8, Xudao 9, Yangyujing 2, Huajing 5, Yandao 16, respectively;

FIG. 9A ALS628W functional marker detection of F2 isolated population individual plant (partial) (ALS4): M is DL2000 molecular marker, 1 is Xudao 9, 2 is Nanjing 9108 mutant, 3 is Xudao 9/Nanjing 9108 mutant, and 1-21 are F2 of Xudao 9/Nanjing 9108 mutant individual plant. R is an herbicide resistant, and S is herbicide susceptibleness;

FIG. 9B ALS628W functional marker detection of F2 isolated population individual plant (partial) (ALS6): M is DL2000 molecular marker, 1 is Xudao 9, 2 is Nanjing 9108 mutant, 3 is Xudao 9/Nanjing 9108 mutant, and 1-21 are F2 of Xudao 9/Nanjing 9108 mutant individual plant. R is an herbicide resistant, and S is herbicide susceptibleness.

### DETAILED DESCRIPTION OF EMBODIMENTS

The embodiments of the invention will be described in detail below in combination with examples, but those skilled in the art will understand that the following examples are only used to illustrate the present invention and should not be regarded as limiting the scope of the present invention. If specific conditions are not indicated in the examples, the routine conditions or the conditions recommended by the manufacturer are used. The reagents or instruments used without the manufacturer's indication are all conventional products that are commercially available.

The background variety selected in the present invention is Nanjing 9108 (purchased from Jiangsu Gaoke Seed Industry Co., Ltd.), which is a new late-maturing medium japonica cultivar selected by the Jiangsu Academy of Agricultural Sciences for research on food crops, with the full growth period of about 150 days. It is suitable for planting in the Central Jiangsu area and Ningzhenyang hilly area of Jiangsu Province. It has been widely used in production and is well received by the market with excellent comprehensive agronomic traits. Nanjing 9108 has a compact plant type, strong tillering ability, strong lodging resistance, good maturity, amylose content of about 10%, and rice appearance is cloudy and fragrant. It is not resistant to imidazolinone herbicides. The invention uses the CRISPR/Cas9 gene editing technology to perform site-specific editing on the Nanjing 9108 ALS gene to obtain a mutant resistant to imidazolinone herbicides, so as to meet the urgent needs of simple cultivation and production.

### Example 1: Obtaining Process of Rice Mutant Resistant to Imidazolinone Herbicides (Imazethapyr)

1. ALS Gene Cloning and the Target Site Design in Nanjing 9108

The genomic DNA of Nanjing 9108 was extracted by the CTAB method as Murray et al. (Murray M G, et al., Nucleic Acids Research, 1980, 8(19): 4321-4326). The genomic DNA was amplified by PCR using primers ALSS-F: TCGCCCAAACCCAGAAACCC (SEQ ID NO: 10), ALSS-R: CTCTTTATGGGTCATTCAGGTC (SEQ ID NO: 11) to obtain amplified products which was send to Invitrogen (Shanghai) Trading Co., Ltd. for sequencing. Sequencing results were performed on NCBI (https://blast.ncbi.nlm.nih.gov/Blast.cgi) database for Blast comparison analysis, and it was found that the ALS coding region sequence of Nanjing 9108 was identical to that of the reference genome rice Nipponbare.

According to the ALS gene sequence of Nanjing 9108, 5′-TCCTGAATGCGCCCCCACT-3′ (SEQ ID NO: 26) was selected as the target site for gene editing by prediction on the CRISPR-GE website (http://skl.scau.edu.cn/targetdesign/). The Cas9 cleavage site caused by this target site is located between 1881 bp position and 1882 bp position, and the variation of adjacent bases is expected to cause amino acid variation at position 627 or 628, to obtain a new herbicide resistant genotype.

2. Construction of CRISPR/Cas9 Gene Editing Vector

The construction of gene editing vector referred to the method reported by Mao et al. (Mao Y, et al., Mol Plant, 2013, 6(6): 2008-2011.), and proceeded as follows:

### (1) Preparation of a Target Adaptor

Adaptor primers (ALS-U3-F: 5′-ggcaTCCTTGAATGCGCCCCCACT-3′(SEQ ID NO: 12); ALS-U3-R: 5′-aaacAGTGGGGGCGCATTCAAGGA-3′(SEQ ID NO: 13)) was dissolved by 1×TE (PH8.0) to obtain 100 μM stock solutions, both pipetted 1 μl from which was added into 98 μl 0.5×TE mix to 1 μM. The products were placed at 90° C. for 30 s and then cooled at room temperature to complete annealing, thereby obtaining the target adaptor

(2) Preparation of sgRNA Expression Cassette

PCR amplification was performed according to the following reaction system:

| Components | Volume | |
|---|---|---|
| pYLsgRNA-OsU3 (10 ng) | 1 | μl |
| Target adaptor | 1 | μl |
| 10 × DNA ligase buffer | 1 | μl |
| Bsal (5 U/μl) | 0.5 | μl |
| T4 DNA ligase (35 U/μl) | 0.2 | μl |
| ddH₂O | Up to 10 | μl |

Note:
T4 DNA ligase and 10 × DNA ligase buffer were purchased from Takara, and Bsal was purchased from NEB.

The PCR reaction program was as follows: 37° C. 5 min, 20° C. 5 min, 5 cycles. The PCR products obtained were the sgRNA ligation products.

pYLsgRNA-OsU3 was an intermediate vector that provided a promoter and guide sequence skeleton for the sgRNA expression cassette. It was developed by the team of Professor Yaoguang Liu from South China Agricultural University (Ma X, Zhang Q Zhu Q et al. A robust CRISPR/Cas9 system for convenient, high-efficiency multiplex genome editing in monocot and dicot plants. Mol Plant, 2015, 8(8): 1274-1284.).

(3) sgRNA Expression Cassette Amplification

PCR amplification was performed according to the following reaction system by a primer combination of forward primer U-F: 5′-CTCCGTTTTACCTGTGGAATCG-3′ (SEQ ID NO: 14) and reverse primer gRNA-R: 5′-CGGAGGAAAATTCCATCCAC-3′(SEQ ID NO: 15):

| Components | Volume | |
|---|---|---|
| 2 × PrimeSTAR GC Buffer | 7.5 | μl |
| dNTP Mix | 1.5 | μl |
| U-F (0.2 μM) | 1.5 | μl |
| gRNA-R (0.2 μM) | 1.5 | μl |
| PrimeSTAR HS DNA Polymerase | 0.2 | μl |
| (2.5 U/μl) | | |
| sgRNA ligation products | 2 | μl |
| ddH₂O | Up to 15 | μl |

Wherein, PrimeSTAR HS DNA Polymerase, dNTP Mix and 2×PrimeSTAR GC Buffer were all purchased from Takara. PCR was performed in an Eppendorf Mastercycle thermal cycler. The PCR reaction program was as follows: 95° C. 1 min; 95° C. 10 s, 60° C. 15 s, 68° C. 20 s, 10 cycles; 95° C. 10 s, 60° C. 15 s, 68° C. 30 s, 22 cycles; stored at 4° C.

PCR amplification was performed according to the following reaction system by amplification primers of Uctcg-B1′: TTCAGAggtctcTctcgCACTGGAATCGGCAGCAAAGG-3 (SEQ ID NO: 16); gRcggt-BL: AGCGTGggtctcGaccgGGTCCATCCACTCCAAGCTC-3 (SEQ ID NO: 17):

| Components | Volume | |
|---|---|---|
| 2 × PrimeSTAR GC Buffer | 10 | μl |
| dNTP Mix | 2 | μl |
| Uctcg-B1′ (2 μM) | 1.5 | μl |
| gRcggt-BL (2 μM) | 1.5 | μl |
| PrimeSTAR HS DNA | 0.25 | μl |
| Polymerase (2.5 U/μl) | | |
| PCR products diluted 50 times | 2 | μl |
| in the first cycle | | |
| ddH₂O | Up to 20 | μl |

PCR was performed in an Eppendorf Mastercycle thermal cycler. The PCR reaction program was as follows: 95° C. 10 s, 60° C. 15 s, 68° C. 20 s, 25 cycles; stored at 4° C. The PCR products obtained were the sgRNA expression cassette.

(4) Ligating the sgRNA Expression Cassette to the pYLCRISPR/Cas9P35S-H Vector

The sgRNA expression cassette was ligated to the pYLCRISPR/Cas9P35S-H vector to obtain ligation products according to the following reaction system and process, in the Eppendorf Mastercycle thermal cycler.

### Reaction System and Process:

| Components | Volume | |
|---|---|---|
| sgRNA expression cassette | 3 | μl |
| pYLCRISPR/Cas9P_{35S}-H | 5 | μl |
| vector | | |
| Bsal (10 U/μl) | 1 | μl |

37° C. 10 min

| | | |
|---|---|---|
| 10 × DNA ligase buffer | 2 | μl |
| T4 DNA ligase (35 U/μl) | 0.5 | μl |
| ddH₂O | Up to 15 | μl |

37° C. 5 min, 10° C. 5 min, 20° C. 5 min, 15 cycles

The pYLCRISPR/Cas9P35S-H vector was a plant binary expression vector developed by the team of Professor Liu Yaoguang of South China Agricultural University (Ma X, Zhang Q Zhu Q et al. A robust CRISPR/Cas9 system for convenient, high-efficiency multiplex genome editing in monocot and dicot plants. Mol Plant, 2015, 8(8): 1274-1284.)

(5) Transformation of E. coli DH5α and Verification

The ligation products were transformed into E. coli by heating shock method (42° C.), and the bacterial solution was spread on an LB plate containing 50 mg/I kanamycin and cultured for about 12 h. Pick a single colony grown on the plate and shake the bacteria to propagate. The bacterial solution was used as a template for PCR verification.

The PCR reaction system was:

| Components | Volume | |
|---|---|---|
| 10 × Taq Buffer | 2 | μl |
| (containing 20 mM Mg²⁺) | | |
| dNTP Mix | 2 | μl |
| Forward primer (2 μM) | 2 | μl |
| Reverse primer (2 μM) | 2 | μl |
| Taq DNA Polymerase | 0.5 | μl |
| (2.5 U/μl) | | |
| Bacteria solution | 2 | μl |
| ddH₂O | Up to 20 | μl |

Taq DNA polymerase was purchased from Beijing Dingguo Changsheng Biotechnology Co., Ltd.

PCR was performed in an Eppendorf Mastercycle thermal cycler. PCR reaction program was as follows: 95° C. 10 min; 95° C. 30 s, 51° C. 30 s, 72° C. 45 s, 28 cycles; 72° C. 5 min; stored at 4° C. The amplified products were separated by agarose gel electrophoresis, photographed with a gel imager and the results were recorded. The plasmids of the bacterial solution containing the target bands were extracted by PCR and sent to Invitrogen (Shanghai) Trading Co., Ltd. for sequencing.

### (6) Obtaining Herbicide Resistant Mutants

The above positive plasmid was transformed into Agrobacterium EHA105. The conventional Agrobacterium-mediated method was used to transform rice Nanjing 9108 (purchased from Jiangsu Gaoke Seed Industry Co., Ltd.). In order to increase the probability of obtaining resistant plants and obtain as many transformed plants as possible, the present invention obtained 58 transgenic plants (T₀ generation) in total.

The T₀ generation plants were harvested as an individual plant, and the harvested seeds were germinated and sown at a density of 450 kg hm⁻². When the rice growed to two leaves and one heart, the water was drained from the field and imazethapyr (aqueous agent, purchased from Nanjing Aijin Agrochemical Co., Ltd.) was sprayed at 210 g (a.i.) hm⁻². Rehydrate after spraying for 24 h, and investigate resistance after 14 days. Plants with all withered or dead leaves were susceptibleness, while the healthy and viable plants were resistance. Among the 58 lines (A1-A58), only 18 of the A51 lines survived and showed herbicide resistance, and all the other 57 lines died (FIG. 2). Therefore, the frequency of obtaining herbicide resistant strains in this study was 1.72%. According to reports, the mutation frequency of herbicide resistant rice lines obtained by chemical mutagenesis was 0.00003^{˜}0.006%. Therefore, the gene editing breeding method used in the present invention was more than 285 times more efficient than chemical mutagenesis to obtain herbicide resistant strains, which was significantly better than chemical mutagenesis.

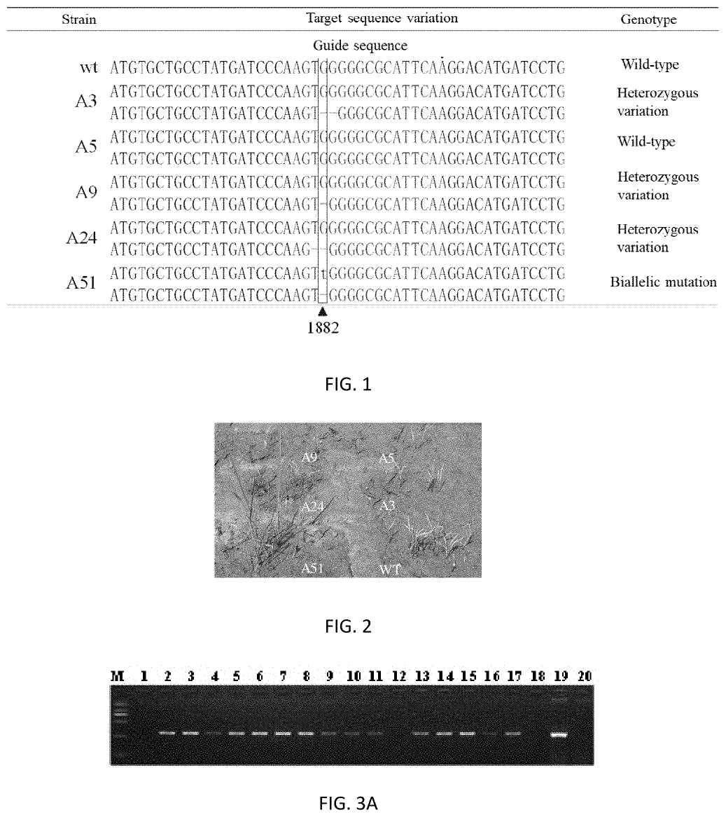

In order to identify the base variation of the editing site, primers ALST-F: CGCATACATACTTGGGCAAC(SEQ ID NO:) and ALST-R: ACAAACATCATAGGCATACCAC(SEQ ID NO:) were used to amplify and sequence part of the T₀ generation transgenic lines. The results were shown in FIG. 1, wherein A5 was not mutated; A3, A9 and A24 were heterozygous mutations. One allele of these individual plants was not mutated, and the other allele was deleted 1 or 2 bases and a frameshift mutation occurred.; A51 was a biallelic mutation, one of which was G to T mutation at 1882 bp position, and the other allele was G base deletion at 1882 bp position (FIG. 1).

### Example 2: Cloning of ALS Gene of Rice Mutant Resistant to Imidazolinone Herbicide

The 18 individual plants of the T₁ generation of the A51 strain of the above-mentioned Example 1 were numbered, their plant leaves were taken, genomic DNA was extracted, and the ALS gene full-length specific primers ALS-F5T-TCGCCCAAACCCAGAAACCC-3T (SEQ ID NO: 10) and ALS-R 5′-CTCTTTATGGGTCATTCAGGTC-3′ (SEQ ID NO: 11) for PCR amplification. The amplified products were sent to Invitrogen (Shanghai) Trading Co., Ltd. for sequencing. The sequencing results were compared with the wild-type ALS gene of Nanjing 9108, and it was found that the individual plants numbered 1-9, 11, 14, 16 and 17 were homozygous mutations from G to T at 1882 bp position; the individual plants numbered 10, 12, 13 and 15 had biallelic mutations. One of the alleles had mutation from G to T at 1882 bp position, and the other allele was G base deletion at 1882 bp position; no homozygous individual plant with deletion of base at 1882 bp position, it was speculated that the frameshift mutant rice with the base deletion could not survive. The homozygous mutations or heterozygous mutations from G to T at 1882 bp position were all resistant to herbicides. It was speculated that this mutation was the key mutation for herbicide resistance.

We further analyzed the mutation from G to T at 1882 bp portion of the ALS gene in the herbicide resistant rice mutant, resulting in the amino acid mutation at position 628 from glycine to tryptophan. The nucleotide sequence of the ALS gene of the herbicide resistant mutant was shown in SEQ ID NO: 1, and the amino acid sequence of the encoded ALS protein was shown in SEQ ID NO: 2, and the cloned new gene was named ALS-nj.

The mutation of base 1882 of the ALS-nj gene identified in the present invention from wild-type G to T, and the mutation of amino acid at position 628 from glycine to tryptophan caused by this were reported for the first time.

### Example 3 T-DNA Deletion of Rice Mutant Resistant to Imidazolinone Herbicide

The invention constructed a binary T-DNA vector for directed editing of ALS genes, and the T-DNA involved in the invention mainly contained the hygromycin phosphotransferase HPT gene and the Cas9 nuclease gene. Since the main function of the hygromycin phosphotransferase HPT gene and the Cas9 gene was to complete the site-directed mutation of the target gene, and these two genes were foreign genes relative to the rice genome, on the one hand, hygromycin was an antibiotic and needed to be deleted, but if the Cas9 gene was retained, it could also lead to functions such as continued editing; on the other hand, random insertion of T-DNA could also cause unexpected gene mutations, so it needed to be cleared after completion of the task of gene editing. Through Agrobacterium-mediated transformation of Nanjing 9108, the T-DNA sequence would be randomly inserted into the chromosome of rice during the transgenic process, and it could be inserted in single or multiple copies. Since the T-DNA insertion site and its target sites were generally not ligated, it was expected that the offspring of transgenic plants could be separated to obtain plants without T-DNA. Even if they were ligated, varieties without T-DNAcould be screened through genetic exchange recombination. Therefore, in order to obtain T-DNA free plants, the inventors performed simultaneous detection for the HPT gene and Cas9 gene in the T₁ generation plant with double mutations in the target gene, repeated 3 times, and screened that they did not carry these two genes, which was a deletion T₁ generation individual plant of T-DNA.

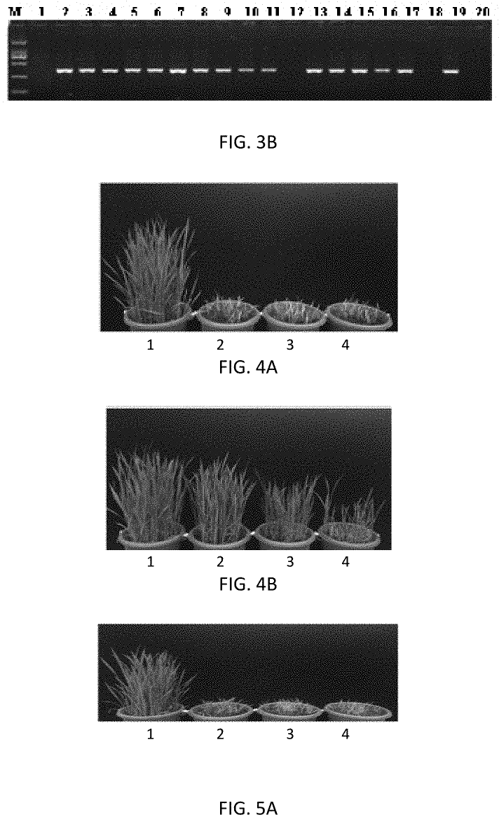

The genomic DNA of the 18 individual plants of the above Example 2 was taken, which was used to PCR amplification for the HPT gene with the primers hyg283-F:TCCGGAAGTGCTTGACATT (SEQ ID NO: 22) and hyg283-R:GTCGTCCATCACAGTTTGC(SEQ ID NO: 23); and for the Cas9 gene with primers Cas9T-F:AGCGGCAAGACTATCCTCGACT (SEQ ID NO: 24) and Cas9T-R:TCAATCCTCTTCATGCTCCC(SEQ ID NO: 25). The results were shown in FIG. 3. The HPT gene and Cas9 gene were not detected in the individual plants numbered 1, 12, and 18, indicating that these three individual plants successfully deleted foreign T-DNA. In this example, 3 of the 18 individual plants had been recombined to delete the T-DNA, and the ratio of the deleted T-DNA plants was one-sixth. It was assumed that the T-DNA was inserted into the rice genome in multiple copies.

### Example 4 Identification of Resistance of Mutant A51 to Imazethapyr (Imidazolium, Imidazolinone Herbicide)

The homozygous mutant T₁ individual plants identified in Examples 2 and 3 were propagated, and the seeds were harvested in the artificial climate room of Nanjing Jiangsu Academy of Agricultural Sciences, which were the T₂ generation; the T₂ generation was continued to be propagated to obtain T₃ Generation seeds. After accelerating the germination of the harvested T₃ seeds, they were sown at a density of 450 kg hm⁻². When the rice growed to two leaves and one heart, the field water was drained and imazethapyr (aqueous agent, purchased from Nanjing Aijin Agrochemical Co., Ltd.) was sprayed at 210, 700, and 1400 g(a.i.) hm⁻² concentrations, respectively, with the water as the control group. After spraying for 24 h, the water was rehydrated, and the resistance was investigated 14 days later. As shown in FIG. 4, both wild-type and mutants could grow normally in the control group sprayed with water; wild-types all died under treatments with concentrations of 210, 700, and 1400 g g(a.i.) hm⁻² imazethapyr; The mutants could survive under treatments with concentrations of 210, 700, and 1400 g(a.i.) hm⁻² imazethapyr. The above results indicated that the mutants were resistant to imazethapyr herbicide and could be stably passed on to the next generation.

### Example 5 Identification of Resistance of Mutant A51 to Bailongtong (Imazameth, Imidazolinone Herbicide)

After accelerating germination of the T₃ generation seeds in Example 4, they were sown at a density of 450 kg hm⁻². When the rice growed to two leaves and one heart, water in the field water was drained and Bailongtong (aqueous agent, purchased from Nanjing Aijin Agrochemical Co., Ltd.) was sprayed at 240, 2400, 4800 g(ai) hm⁻² concentration with water as the control group. After spraying for 24 h, the water was rehydrated, and the resistance was investigated 14 days later. As shown in FIG. 5, both the wild type and the mutant could grow normally in the control group sprayed with water; the wild type died after treatment with Bailongtong at the concentration of 240, 2400, 4800 g(ai) hm⁻²; The mutants survived under treatments with concentrations of 240 and 2400 g(ai) hm⁻² imazethapyr, and died after treatments with Bailongtong at 4800 g(ai) hm⁻². The above results indicate that the mutant can resist a concentration of 2400 g (a.i.) hm⁻² methimazole nicotinic acid, and its resistance is stably passed on to the next generation.

### Example 6: Investigation of Agronomic Traits of Mutants

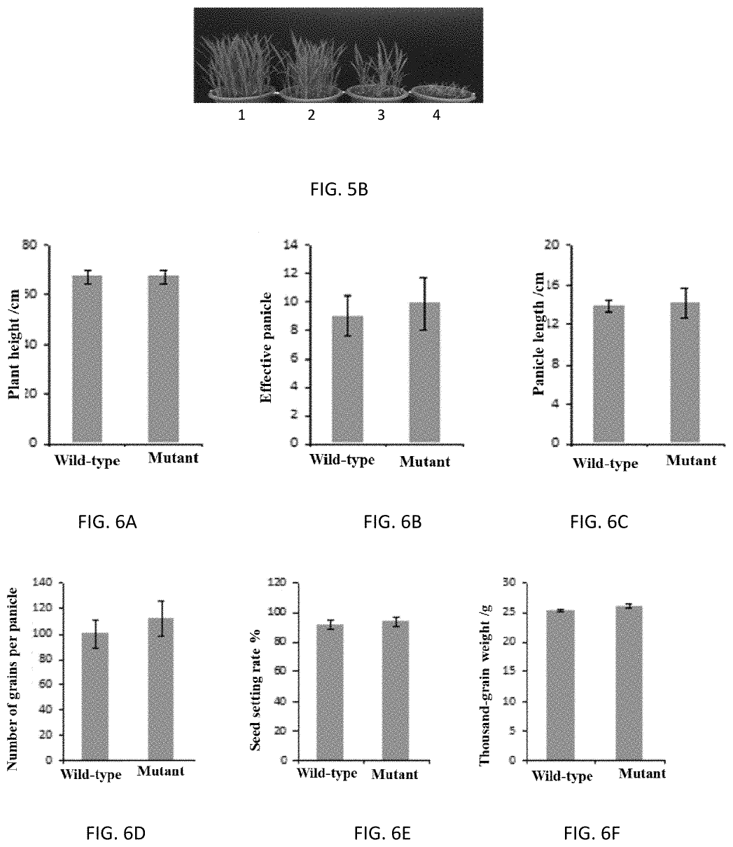

The wild-type and homozygous mutants with T-DNA deletion identified in Examples 2 and 3 were planted in the experimental base of Sanya City, Hainan Province. The wild-type and mutant were planted in plots with 200 seedlings per plot, repeated three times. The analysis of agronomic traits showed that the wild-type and mutants were compared with six yield components including plant height, effective panicle, panicle length, number of grains per panicle, seed setting rate, and thousand-grain weight. The T test showed that the difference was not significant (P<0.05) (FIG. 6). There were no significant differences in other agronomic traits such as heading date, plant leaf morphology, leaf color, rice aroma, rice appearance (cloudy) and other agronomic characteristics. Therefore, the new herbicide-resistant variety obtained by editing retained important agronomic characteristics such as high yield and high quality of wild-type variety.

### Example 7: Development and Use of Genetic Characteristics of ALS-Nj Gene and Functional Markers Thereof

Molecular marker assisted selection was beneficial to speed up the breeding process. The ALS-nj gene of the present invention was a single base mutation, which could be specifically designed for enzyme digestion target markers, but the process was relatively cumbersome. The allele-specific PCR was developed, which could distinguish resistant genotypes through two cycles of PCR, and the operation of which was simple and fast. In the present invention, 13 sets of primers ALS1 to ALS13 (Table 1) were designed based on the base variation of wild-type and mutant at 1882 bp position of the ALS gene, using the principle of allele-specific PCR. ALS1˜ALS7 shared the forward primer ALS-1F, and the reverse primers were ALS-1R, ALS-2R, ALS-3R, ALS-4R, ALS-5R, ALS-6R and ALS-7R. ALS7-ALS13 shared the reverse primer ALS-1R, and the forward primers were ALS-2F, ALS-3F, ALS-4F, ALS-5F, ALS-6F and ALS-7F, respectively. In order to further improve the specificity of primers, a base mismatch was introduced at the 3′ terminal of some primers. The third base of ALS-3F and ALS-6F primers from 3′ to 5′ direction was mismatched from G to A, the third base of ALS-4F and ALS-7F primers from 3′ to 5′ direction was mismatched from G to C, the third base of ALS-3R and ALS-6R primers from 3′ to 5′ direction was mismatched from C to T, and the third base of ALS-4R and ALS-7R primers from 3′ to 5′ direction was mismatched from C to A.

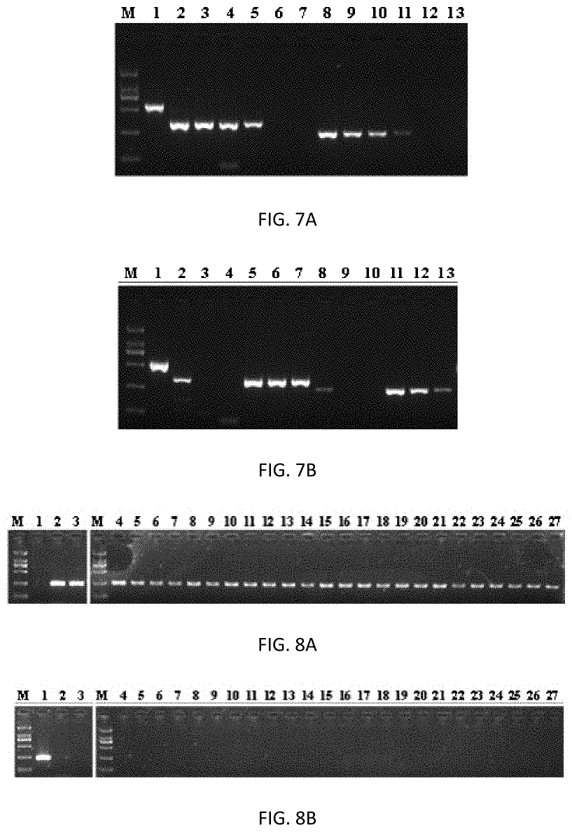

After multiple rounds of screening and optimization of PCR reaction conditions, it was found that the primer sets ALS4 and ALS6 had excellent amplification efficiency and specificity, which could be used as primer sets to distinguish wild-type, mutant genotype, and heterozygous genotype respectively (FIG. 7). The best reaction system for PCR was: wild-type or mutant DNA template 2 μL, 10×PCR buffer 2 μL, MgCl₂ (5 mmol/L) 2 μL, dNTP (2 mmol/L) 2 μL, the forward primer 2 μL, the reverse primer 2 μL, Taq enzyme (2.5 U/μl) 0.2 μL, ddH₂O 7.8 μL. The PCR reaction program was 95° C. 10 min; 95° C. 30 s, 60° C. 30 s, 72° C. 45 s, 35 cycles; 72° C. 5 min; stored at 4° C. The molecular marker composed of ALS4 and ALS6 was named ALS628W.

TABLE 1
Molecular markers for detecting mutant genes

| Marker name | Primer name | Primer sequence (5′→3′) |
|---|---|---|
| ALS1 | ALS-1F | ATCCGCATTGAGAACCTCC (SEQ ID NO: 6) |
| | ALS-1R | TAGGATTACCATGCCAAGCAC (SEQ ID NO: |
| | | 27) |
| ALS2 | ALS-1F | ATCCGCATTGAGAACCTCC (SEQ ID NO: 6) |
| | ALS-2R | ATGTCCTTGAATGCGCCCCC (SEQ ID NO: |
| | | 29) |
| ALS3 | ALS-1F | ATCCGCATTGAGAACCTCC (SEQ ID NO: 6) |
| | ALS-3R | ATGTCCTTGAATGCGCCtCC (SEQ ID NO: |
| | | 28) |
| ALS4 | ALS-1F | ATCCGCATTGAGAACCTCC (SEQ ID NO: 6) |
| | ALS-4R | ATGTCCTTGAATGCGCCaCC (SEQ ID NO: |
| | | 20) |
| ALS5 | ALS-1F | ATCCGCATTGAGAACCTCC (SEQ ID NO: 6) |
| | ALS-5R | ATGTCCTTGAATGCGCCCCA (SEQ ID NO: |
| | | 21) |
| ALS6 | ALS-1F | ATCCGCATTGAGAACCTCC (SEQ ID NO: 6) |
| | ALS-6R | ATGTCCTTGAATGCGCCtCA (SEQ ID NO: |
| | | 30) |
| ALS7 | ALS-1F | ATCCGCATTGAGAACCTCC (SEQ ID NO: 6) |
| | ALS-7R | ATGTCCTTGAATGCGCCaCA (SEQ ID NO: |
| | | 31) |
| ALS8 | ALS-2F | TGCTGCCTATGATCCCAAGTG (SEQ ID NO: |
| | | 32) |
| | ALS-1R | TAGGATTACCATGCCAAGCAC (SEQ ID |
| | | NO:) |
| ALS9 | ALS-3F | TGCTGCCTATGATCCCAAaTG (SEQ ID NO: |
| | | 33) |
| | ALS-1R | TAGGATTACCATGCCAAGCAC (SEQ ID NO: |
| | | 27) |
| ALS10 | ALS-4F | TGCTGCCTATGATCCCAAcTG (SEQ ID NO: |
| | | 34) |
| | ALS-1R | TAGGATTACCATGCCAAGCAC (SEQ ID NO: |
| | | 27) |
| ALS11 | ALS-5F | TGCTGCCTATGATCCCAAGTT (SEQ ID NO: |
| | | 35) |
| | ALS-1R | TAGGATTACCATGCCAAGCAC (SEQ ID NO: |
| | | 27) |
| ALS12 | ALS-6F | TGCTGCCTATGATCCCAAaTT (SEQ ID NO: |
| | | 36) |
| | ALS-1R | TAGGATTACCATGCCAAGCAC (SEQ ID NO: |
| | | 27) |
| ALS13 | ALS-7F | TGCTGCCTATGATCCCAAcTT (SEQ ID NO: |
| | | 37) |
| | ALS-1R | TAGGATTACCATGCCAAGCAC (SEQ ID NO: |
| | | 27) |

Note:
Bases marked in lowercase letters are mismatched bases.

The ALS628W marker was used to detect rice varieties, and it was found that among the tested varieties, only the Nanjing 9108 mutant could be amplified by ALS6, and the remaining japonica or indica varieties (Nanjing 9108 wild type, Nipponbare, Huang Huazhan, 9311, Lianjing 7, Suxiu 867, Zhendao 88, Zhendao 99, Huaidao 5, Changnongjing No. 8, Nanjing 44, Nanjing 45, Nanjing 46, Nanjing 49, Nanjing 51, Nanjing 47, Nanjing 5055, Suken 118, Wuyunjing 24, Wuyunjing 27, Wuyunjing 29, Xudao 8, Xudao 9, Yangyujing 2, Huajing 5, Yandao 16) could only be amplified to obtain bands by ALS4 (FIG. 8), indicating that the ALS628W marker could specifically detect the mutation from G to T at position 1882 of the ALS-nj gene. This marker could be used for molecular marker-assisted selection breeding.

In order to verify the use of the ALS628W marker in the selection of herbicide resistant strains, the ALS628W marker was further used to detect Xudao 9, Nanjing 9108 mutant, “Xudao 9/Nanjing 9108 mutant” hybrids and 132 of them F₂ individual plants, and identify their phenotypes. The hybrid “Xudao 9/Nanjing 9108 mutant” showed herbicide resistance. Among F₂ individual plants, 102 individual plants showed herbicide resistance and 30 individual plants showed herbicide susceptibility, the separation ratio of which conformed to 3:1 (χ2=0.2525, P>0.05) by Chi-square test.

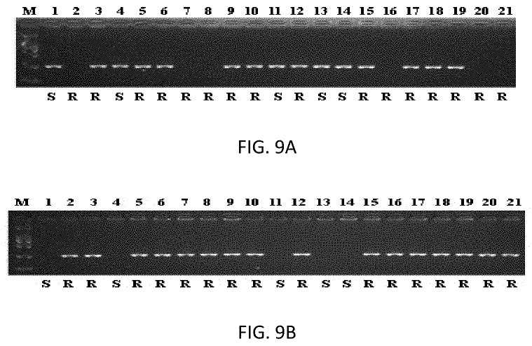

Based on the ratio of resistance to susceptible parents, hybrid F₁ and F₂, the herbicide resistance of ALS-nj gene was a dominant trait controlled by a single gene. Combined with the results of the marker detection, it was found that all F₂ populations with mutant genes were resistant to herbicides, and all F₂ populations without mutant genes were resistant to herbicides (FIG. 9). The genotype test results corresponded exactly to the phenotype identification results. The ALS628W marker was completely co-segregated with the resistant/susceptible herbicide phenotype, and the resistant heterozygous genotype could be detected at the same time, indicating that the ALS628W marker developed by us could be used for precise breeding of herbicide resistance such as imazethapyr, and the homozygous genotypes for herbicide resistance could be screened in the F₂ generation, which could be used for early generation selection.

The above specific examples showed that the use of CRISPR/Cas9 gene editing technology to edit the ALS gene, a new variety with stable inheritance of T-DNA deletion and herbicide resistance could be obtained in the T₂ generation through the screening of offspring, but the basic agronomic characteristics of the new variety had not changed significantly. Compared with chemical mutagenesis, cross-breeding and other breeding methods, gene editing directed improvement molecular breeding technology is fast, accurate and efficient, combined with gene function marker genotype selection, would greatly improve breeding efficiency and greatly accelerate the breeding process (Table 2).

TABLE 2
Comparison of the gene editing breeding method
and the traditional breeding method

| Traditional breeding | Gene editing breeding |
|---|---|
| method | method |
| It tooke 6 to 8 years for | It only took 1-2 years |
| backcrossing 5-6 | for vector construction |
| generations and self-bringing | and transgene for 3 months, |
| 4-5 generations to obtain | selecting the herbicide |
| a new variety with | resistant strains with |
| stable characteristics; large | T-DNA deleted for 4 months, |
| randomness, difficult chain | and propagation for |
| gene recombination is, | 5-12 months until finally |
| and high labor cost. | obtaining a new variety with |
| | stable characteristics; well |
| | specificity, no effect on |
| | other agronomic traits, no |
| | chain burden, and |
| | low labor costs. |

Gene editing breeding methods can increase
breeding efficiency by 3 to 8 times.

Although the specific embodiments of the present invention have been described in detail, those skilled in the art will understand that according to all the teachings that have been disclosed, various modifications and substitutions can be made to those details, which are all within the protection scope of the present invention. The full scope of the invention is given by the appended patent claims and any equivalents.

What is claimed is:

1. A rice ALS mutant protein, wherein a mutation corresponding to an amino acid at position 628 of the amino acid sequence of the rice ALS is present in the amino acid sequence of the rice ALS protein, the amino acid at position 628 is mutated from glycine to tryptophan.

2. The rice ALS mutant protein according to claim 1, an amino acid sequence of which is shown in SEQ ID NO: 2.

3. The rice ALS mutant protein according to claim 1, the amino acid sequence is encoded by a nucleic acid or gene.

4. The rice ALS mutant protein according to claim 3, wherein:
(a) the nucleic acid or gene encoding the mutant protein of claim 1; or
(b) having a nucleotide sequence as shown in SEQ ID NO:1.

5. The rice ALS mutant protein according to claim 3, wherein the nucleic acid or gene is inserted in an expression cassette.

6. The rice ALS mutant protein according to claim 5, wherein the expression cassette is transformed into a plant.

7. The rice ALS mutant protein according to claim 3, wherein an herbicide resistant plant is prepared by the following steps:
1) transforming the nucleic acid or gene of claim 3 into a plant; or
2) making the plant express the rice ALS mutant protein having the amino acid sequence showed as SEQ ID NO: 2.

8. A breeding method for creating herbicide resistant rice using gene editing, wherein the method comprises the following steps:
1) cloning ALS gene and designing a target site for gene editing;
2) construction of CRISPR/Cas9 gene editing vector containing a target fragment;
3) obtaining herbicide resistant rice expressing a mutant protein having an amino acid sequence showed as SEQ ID NO: 2.

9. The breeding method according to claim 8, wherein the nucleotide sequence of the target sites for gene editing in step 1) is shown in SEQ ID NO:5.

10. The breeding method of claim 8, wherein the CRISPR/Cas9 gene editing vector containing the target fragment in the step 2) is constructed as follows:
a) preparation of a target adaptor: a adaptor primer is dissolved with TE to obtain a stock solution, which is placed at 90° C. for 30 s after dilution and then cooled at room temperature to complete annealing, thereby obtaining the target adaptor;
b) preparation of an sgRNA ligation product: the sgRNA ligation product is obtained by PCR amplification using a pYLsgRNA-OsU3 intermediate vector, the target adaptor, DNA ligase, and BsaI;
c) sgRNA expression cassette amplification: the sgRNA ligation product is subjected to the first cycle of PCR amplification by a primer combination of forward primer U-F and reverse primer sgRNA-R to obtain the first cycle of PCR product, which is then subjected to the second cycle of PCR after dilution by Uctcg-B1 and gRcggt-BL as amplification primers to obtain a PCR product which is the sgRNA expression cassette;
d) the sgRNA expression cassette is ligated to a CRISPR/Cas9 expression vector to obtain a ligation product;
e) the ligation product from step d) is transformed into E. coli by heating shock to obtain recombinant bacteria, and positive plasmids are extracted from the verified bacterial solution containing a target brand.

11. The breeding method according to claim 8, wherein the method of the step 3) is as follows: the CRISPR/Cas9 gene editing vector containing the target fragment from the step 2) is transformed into Agrobacterium EHA105 to obtain a T₀ generation transgenic plant with herbicide resistance, for which the sequence is amplified and identified by primers ALST-F and ALST-R to obtain a plant with the mutant protein of claim 1.

12. The breeding method according to claim 11, wherein the breeding method further comprises deleting a T-DNA vector including the hygromycin phosphotransferase gene HPT and the nuclease gene Cas9 from a T₁ generation plant containing the target allele double mutation of the T₀ generation transgenic plant with herbicide resistance.

13. The breeding method according to claim 12, wherein the deletion of the T-DNA vector involves simultaneous detection for the HPT gene and the Cas9 gene in the T₁ generation plant containing the target allele double mutation, which is repeated for multiple times to obtain a T₁ generation individual plant without the two genes after screening, referred as a target plant.

14. The breeding method according to claim 12, wherein the detection for HPT gene involves PCR amplification using the genomic DNA of the T₁ generation plant with the target allele double mutation as a template, and hyg283-F and hyg283-R as primers, and meanwhile, the detection for Cas9 gene involves PCR amplification using the genomic DNA of the T₁ generation plant with the target allele double mutation as a template, and Cas9T-F and Cas9T-R as primers, and absence of both the HPT gene and the Cas9 gene indicates that the T-DNA is successfully deleted.

15. A primer set for identifying the gene or nucleic acid of claim 4, wherein the primer set is ALS4 with sequences as shown in SEQ ID NO: 6 and SEQ ID NO: 7, and/or ALS6 with sequences as shown in SEQ ID NO: 8 and SEQ ID NO: 9.

16. The primer set according to claim 15, wherein the primer set is used for identifying and breeding of an herbicide resistant variety.


SEQUENCE LISTING (37 sequences)

SEQ ID NO: 1 - DNA, 1935 bp, Oryza sativa
atggctacgaccgccgcggccgcggccgccgccctgtccgccgccgcgacggccaagaccggccgtaagaaccaccagcgacaccacgtccttcccgctcgaggccgggtgggggcggcggcggtcaggtgctcggcggtgtccccggtcaccccgccgtccccggcgccgccggccacgccgctccggccgtgggggccggccgagccccgcaagggcgcggacatcctcgtggaggcgctggagcggtgcggcgtcagcgacgtgttcgcctacccgggcggcgcgtccatggagatccaccaggcgctgacgcgctccccggtcatcaccaaccacctcttccgccacgagcagggcgaggcgttcgcggcgtccgggtacgcgcgcgcgtccggccgcgtcggggtctgcgtcgccacctccggccccggggcaaccaacctcgtgtccgcgctcgccgacgcgctgctcgactccgtcccgatggtcgccatcacgggccaggtcccccgccgcatgatcggcaccgacgccttccaggagacgcccatagtcgaggtcacccgctccatcaccaagcacaattaccttgtccttgatgtggaggacatcccccgcgtcatacaggaagccttcttcctcgcgtcctcgggccgtcctggcccggtgctggtcgacatccccaaggacatccagcagcagatggccgtgccggtctgggacacctcgatgaatctaccagggtacatcgcacgcctgcccaagccacccgcgacagaattgcttgagcaggtcttgcgtctggttggcgagtcacggcgcccgattctctatgtcggtggtggctgctctgcatctggtgacgaattgcgctggtttgttgagctgactggtatcccagttacaaccactctgatgggcctcggcaatttccccagtgacgacccgttgtccctgcgcatgcttgggatgcatggcacggtgtacgcaaattatgccgtggataaggctgacctgttgcttgcgtttggtgtgcggtttgatgatcgtgtgacagggaaaattgaggcttttgcaagcagggccaagattgtgcacattgacattgatccagcagagattggaaagaacaagcaaccacatgtgtcaatttgcgcagatgttaagcttgctttacagggcttgaatgctctgctacaacagagcacaacaaagacaagttctgattttagtgcatggcacaatgagttggaccagcagaagagggagtttcctctggggtacaaaacttttggtgaagagatcccaccgcaatatgccattcaggtgctggatgagctgacgaaaggtgaggcaatcatcgctactggtgttgggcagcaccagatgtgggcggcacaatattacacctacaagcggccacggcagtggctgtcttcggctggtctgggcgcaatgggatttgggctgcctgctgcagctggtgcttctgtggctaacccaggtgtcacagttgttgatattgatggggatggtagcttcctcatgaacattcaggagctggcattgatccgcattgagaacctccctgtgaaggtgatggtgttgaacaaccaacatttgggtatggtggtgcaatgggaggataggttttacaaggcgaatagggcgcatacatacttgggcaacccggaatgtgagagcgagatatatccagattttgtgactattgctaaggggttcaatattcctgcagtccgtgtaacaaagaagagtgaagtccgtgccgccatcaagaagatgctcgagactccagggccatacttgttggatatcatcgtcccgcaccaggagcatgtgctgcctatgatcccaagttggggcgcattcaaggacatgatcctggatggtgatggcaggactgtgtattaa

SEQ ID NO: 2 - protein, 644 aa, Oryza sativa
MATTAAAAAAALSAAATAKTGRKNHQRHHVLPARGRVGAAAVRCSAVSPVTPPSPAPPATPLRPWGPAEPRKGADILVEALERCGVSDVFAYPGGASMEIHQALTRSPVITNHLFRHEQGEAFAASGYARASGRVGVCVATSGPGATNLVSALADALLDSVPMVAITGQVPRRMIGTDAFQETPIVEVTRSITKHNYLVLDVEDIPRVIQEAFFLASSGRPGPVLVDIPKDIQQQMAVPVWDTSMNLPGYIARLPKPPATELLEQVLRLVGESRRPILYVGGGCSASGDELRWFVELTGIPVTTTLMGLGNFPSDDPLSLRMLGMHGTVYANYAVDKADLLLAFGVRFDDRVTGKIEAFASRAKIVHIDIDPAEIGKNKQPHVSICADVKLALQGLNALLQQSTTKTSSDFSAWHNELDQQKREFPLGYKTFGEEIPPQYAIQVLDELTKGEAIIATGVGQHQMWAAQYYTYKRPRQWLSSAGLGAMGFGLPAAAGASVANPGVTVVDIDGDGSFLMNIQELALIRIENLPVKVMVLNNQHLGMVVQWEDRFYKANRAHTYLGNPECESEIYPDFVTIAKGFNIPAVRVTKKSEVRAAIKKMLETPGPYLLDIIVPHQEHVLPMIPSWGAFKDMILDGDGRTVY

SEQ ID NO: 3 - DNA, 1935 bp, Oryza sativa
atggctacgaccgccgcggccgcggccgccgccctgtccgccgccgcgacggccaagaccggccgtaagaaccaccagcgacaccacgtccttcccgctcgaggccgggtgggggcggcggcggtcaggtgctcggcggtgtccccggtcaccccgccgtccccggcgccgccggccacgccgctccggccgtgggggccggccgagccccgcaagggcgcggacatcctcgtggaggcgctggagcggtgcggcgtcagcgacgtgttcgcctacccgggcggcgcgtccatggagatccaccaggcgctgacgcgctccccggtcatcaccaaccacctcttccgccacgagcagggcgaggcgttcgcggcgtccgggtacgcgcgcgcgtccggccgcgtcggggtctgcgtcgccacctccggccccggggcaaccaacctcgtgtccgcgctcgccgacgcgctgctcgactccgtcccgatggtcgccatcacgggccaggtcccccgccgcatgatcggcaccgacgccttccaggagacgcccatagtcgaggtcacccgctccatcaccaagcacaattaccttgtccttgatgtggaggacatcccccgcgtcatacaggaagccttcttcctcgcgtcctcgggccgtcctggcccggtgctggtcgacatccccaaggacatccagcagcagatggccgtgccggtctgggacacctcgatgaatctaccagggtacatcgcacgcctgcccaagccacccgcgacagaattgcttgagcaggtcttgcgtctggttggcgagtcacggcgcccgattctctatgtcggtggtggctgctctgcatctggtgacgaattgcgctggtttgttgagctgactggtatcccagttacaaccactctgatgggcctcggcaatttccccagtgacgacccgttgtccctgcgcatgcttgggatgcatggcacggtgtacgcaaattatgccgtggataaggctgacctgttgcttgcgtttggtgtgcggtttgatgatcgtgtgacagggaaaattgaggcttttgcaagcagggccaagattgtgcacattgacattgatccagcagagattggaaagaacaagcaaccacatgtgtcaatttgcgcagatgttaagcttgctttacagggcttgaatgctctgctacaacagagcacaacaaagacaagttctgattttagtgcatggcacaatgagttggaccagcagaagagggagtttcctctggggtacaaaacttttggtgaagagatcccaccgcaatatgccattcaggtgctggatgagctgacgaaaggtgaggcaatcatcgctactggtgttgggcagcaccagatgtgggcggcacaatattacacctacaagcggccacggcagtggctgtcttcggctggtctgggcgcaatgggatttgggctgcctgctgcagctggtgcttctgtggctaacccaggtgtcacagttgttgatattgatggggatggtagcttcctcatgaacattcaggagctggcattgatccgcattgagaacctccctgtgaaggtgatggtgttgaacaaccaacatttgggtatggtggtgcaatgggaggataggttttacaaggcgaatagggcgcatacatacttgggcaacccggaatgtgagagcgagatatatccagattttgtgactattgctaaggggttcaatattcctgcagtccgtgtaacaaagaagagtgaagtccgtgccgccatcaagaagatgctcgagactccagggccatacttgttggatatcatcgtcccgcaccaggagcatgtgctgcctatgatcccaagtgggggcgcattcaaggacatgatcctggatggtgatggcaggactgtgtattaa

SEQ ID NO: 4 - protein, 644 aa, Oryza sativa
MATTAAAAAAALSAAATAKTGRKNHQRHHVLPARGRVGAAAVRCSAVSPVTPPSPAPPATPLRPWGPAEPRKGADILVEALERCGVSDVFAYPGGASMEIHQALTRSPVITNHLFRHEQGEAFAASGYARASGRVGVCVATSGPGATNLVSALADALLDSVPMVAITGQVPRRMIGTDAFQETPIVEVTRSITKHNYLVLDVEDIPRVIQEAFFLASSGRPGPVLVDIPKDIQQQMAVPVWDTSMNLPGYIARLPKPPATELLEQVLRLVGESRRPILYVGGGCSASGDELRWFVELTGIPVTTTLMGLGNFPSDDPLSLRMLGMHGTVYANYAVDKADLLLAFGVRFDDRVTGKIEAFASRAKIVHIDIDPAEIGKNKQPHVSICADVKLALQGLNALLQQSTTKTSSDFSAWHNELDQQKREFPLGYKTFGEEIPPQYAIQVLDELTKGEAIIATGVGQHQMWAAQYYTYKRPRQWLSSAGLGAMGFGLPAAAGASVANPGVTVVDIDGDGSFLMNIQELALIRIENLPVKVMVLNNQHLGMVVQWEDRFYKANRAHTYLGNPECESEIYPDFVTIAKGFNIPAVRVTKKSEVRAAIKKMLETPGPYLLDIIVPHQEHVLPMIPSGGAFKDMILDGDGRTVY

SEQ ID NO: 5 - DNA, 20 bp, Artificial Sequence
  It is synthesized
tccttgaatgcgcccccact

SEQ ID NO: 6 - DNA, 19 bp, Artificial Sequence
  It is synthesized
atccgcattgagaacctcc

SEQ ID NO: 7 - DNA, 20 bp, Artificial Sequence
  It is synthesized
atgtccttgaatgcgccacc

SEQ ID NO: 8 - DNA, 21 bp, Artificial Sequence
  It is synthesized
tgctgcctatgatcccaaatt

SEQ ID NO: 9 - DNA, 20 bp, Artificial Sequence
  It is synthesized
atgtccttgaatgcgcctca

SEQ ID NO: 10 - DNA, 20 bp, Artificial Sequence
  It is synthesized
tcgcccaaacccagaaaccc

SEQ ID NO: 11 - DNA, 22 bp, Artificial Sequence
  It is synthesized
ctctttatgggtcattcaggtc

SEQ ID NO: 12 - DNA, 24 bp, Artificial Sequence
  It is synthesized
ggcatccttgaatgcgcccccact

SEQ ID NO: 13 - DNA, 24 bp, Artificial Sequence
  It is synthesized
aaacagtgggggcgcattcaagga

SEQ ID NO: 14 - DNA, 22 bp, Artificial Sequence
  It is synthesized
ctccgttttacctgtggaatcg

SEQ ID NO: 15 - DNA, 20 bp, Artificial Sequence
  It is synthesized
cggaggaaaattccatccac

SEQ ID NO: 16 - DNA, 38 bp, Artificial Sequence
  It is synthesized
ttcagaggtctctctcgcactggaatcggcagcaaagg

SEQ ID NO: 17 - DNA, 37 bp, Artificial Sequence
  It is synthesized
agcgtgggtctcgaccgggtccatccactccaagctc

SEQ ID NO: 18 - DNA, 20 bp, Artificial Sequence
  It is synthesized
cgcatacatacttgggcaac

SEQ ID NO: 19 - DNA, 22 bp, Artificial Sequence
  It is synthesized
acaaacatcataggcataccac

SEQ ID NO: 20 - DNA, 20 bp, Artificial Sequence
  It is synthesized
atgtccttgaatgcgccacc

SEQ ID NO: 21 - DNA, 20 bp, Artificial Sequence
  It is synthesized
atgtccttgaatgcgcccca

SEQ ID NO: 22 - DNA, 19 bp, Artificial Sequence
  It is synthesized
tccggaagtgcttgacatt

SEQ ID NO: 23 - DNA, 19 bp, Artificial Sequence
  It is synthesized
gtcgtccatcacagtttgc

SEQ ID NO: 24 - DNA, 22 bp, Artificial Sequence
  It is synthesized
agcggcaagactatcctcgact

SEQ ID NO: 25 - DNA, 22 bp, Artificial Sequence
  It is synthesized
tcaatcctcttcatgcgctccc

SEQ ID NO: 26 - DNA, 19 bp, Artificial Sequence
  It is synthesized
tcctgaatgcgcccccact

SEQ ID NO: 27 - DNA, 21 bp, Artificial Sequence
  It is synthesized
taggattaccatgccaagcac

SEQ ID NO: 28 - DNA, 19 bp, Artificial Sequence
  It is synthesized
atgtccttgaatgcgcctc

SEQ ID NO: 29 - DNA, 20 bp, Artificial Sequence
  It is synthesized
atgtccttgaatgcgccccc

SEQ ID NO: 30 - DNA, 20 bp, Artificial Sequence
  It is synthesized
atgtccttgaatgcgcctca

SEQ ID NO: 31 - DNA, 20 bp, Artificial Sequence
  It is synthesized
atgtccttgaatgcgccaca

SEQ ID NO: 32 - DNA, 21 bp, Artificial Sequence
  It is synthesized
tgctgcctatgatcccaagtg

SEQ ID NO: 33 - DNA, 21 bp, Artificial Sequence
  It is synthesized
tgctgcctatgatcccaaatg

SEQ ID NO: 34 - DNA, 21 bp, Artificial Sequence
  It is synthesized
tgctgcctatgatcccaactg

SEQ ID NO: 35 - DNA, 21 bp, Artificial Sequence
  It is synthesized
tgctgcctatgatcccaagtt

SEQ ID NO: 36 - DNA, 21 bp, Artificial Sequence
  It is synthesized
tgctgcctatgatcccaaatt

SEQ ID NO: 37 - DNA, 21 bp, Artificial Sequence
  It is synthesized
tgctgcctatgatcccaactt